(19) United States
(12) Patent Application Publication
(10) Pub. No.: US 2008/0281291 A1
(43) Pub. Date: Nov. 13, 2008

(54) Drainage/irrigation urethral catheter

(76) Inventors: Claude Tihon, Eden Prairie, MN (US); Neil Nye, Columbia Heights, MN (US)

Correspondence Address: DICKE, BILLIG & CZAJA, PLLC, Suite 2250, 100 South Fifth Street, Minneapolis, MN, 55402, US

(21) Appl. No.: 11/800,704

(22) Filed: May 7, 2007

(51) Int. Cl. A61M 31/00

(52) U.S. Cl. 604/517; 604/523

(57) ABSTRACT

A urethral catheter comprises an elongate tubular shaft having an open proximal end, a sealed distal end a single lumen therein that extends between the proximal and distal ends. A proximal portion of the shaft has a first outer diameter and a distal portion of the shaft has a second outer diameter smaller than the first outer diameter. A plurality of openings are provided in the distal shaft portion. The plurality of openings communicate between an outer surface of the shaft and the single lumen and extend longitudinally from a point near the distal end toward the proximal end. The plurality of openings are located along one side of the shaft and are oriented in a common plane that bisects a longitudinal axis of the shaft. The openings most distant from the distal end of the shaft have a diameter that is smaller than that of the openings closest to the distal end of the shaft.

### BACKGROUND

The present invention generally relates to urethral catheters, and more particularly, to urethral catheters for draining the bladder and irrigating the urethra. Prior to a transurethral urological procedure, urine is typically drained from a patient's bladder using a Foley-type urinary catheter. The Foley catheter is then removed and a treatment catheter for performing the urological procedure is inserted into the urethra. Prior to inserting the treatment catheter, it is common to introduce an anesthetic into the urethra by inserting the tip of a syringe into the urethra at the meatus and injecting the anesthetic into the urethra. This technique does not allow anesthetic to flow evenly into the full length of the urethra. Moreover, anesthetic delivered by this technique tends to leak back out of the urethra. There is a need for a simple and inexpensive urethral catheter that can drain a patient's bladder and irrigate the full length of a patient's urethra in a single catheterization procedure.

### SUMMARY OF THE INVENTION

A urethral catheter comprises an elongate tubular shaft having an open proximal end, a sealed distal end, an outer surface and a single lumen extending between the proximal and distal ends. The tubular shaft has a plurality of openings communicating between the outer surface and the lumen that are longitudinally spaced relative to one another and oriented on one side of the shaft in a common plane that bisects a longitudinal axis of the shaft. The plurality of openings comprising a first set of openings adjacent to the distal end of the shaft, a second set of openings spaced proximally of the first set of openings, and a third set of openings spaced proximally from the second set of openings. The third set of openings is distally spaced from the proximal end of the shaft. The plurality of openings comprise the only openings of the catheter.

### BRIEF DESCRIPTION OF THE DRAWINGS

FIG. 1 is a perspective view of the urethral catheter of the present invention;

FIG. 2 is top plan view of the urethral catheter of FIG. 1; and

FIG. 3 is longitudinal sectional view of the urethral catheter of the present invention diagrammatically positioned in a patient in the supine position.

### DETAILED DESCRIPTION

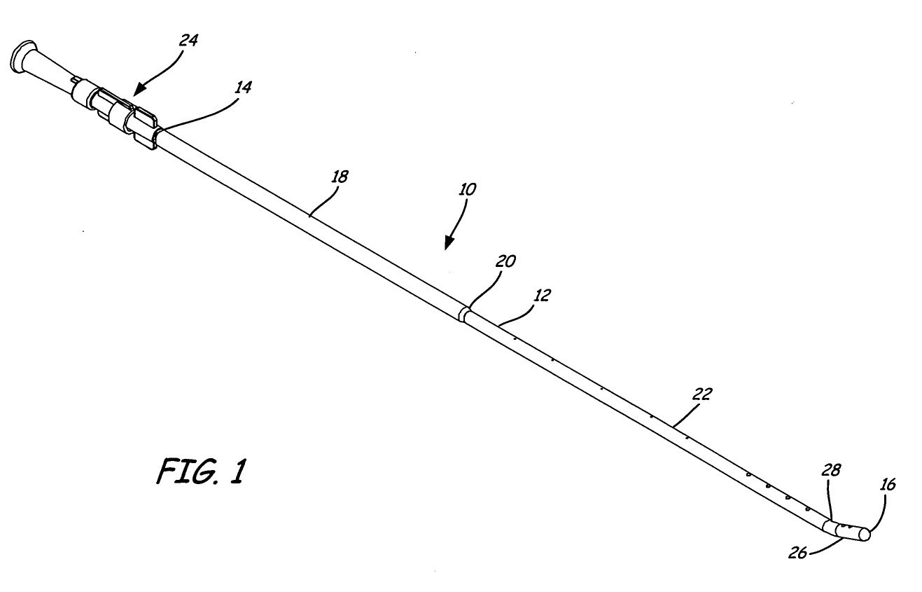

FIG. 1 is a perspective view of catheter 10 of the present invention, which is useful for drainage and irrigation of a body lumen, e.g., the urethra/bladder. Catheter 10 generally comprises an elongate, hollow flexible shaft 12, which extends from an open proximal end 14 to a sealed distal end 16. Shaft 12 may be formed from a suitable biocompatible material, such as thermoplastic polyurethane elastomer or silicone rubber, and may include a hydrophilic coating on the outer surface of the shaft. In one embodiment, shaft 12 is formed from Pellethane 2363-80AE. Shaft 12 may have an outer diameter ranging from 8 to 24 French (i.e., 2.7 mm to 8.0 mm).

In one embodiment, shaft 12 has a proximal shaft portion 18, which extends from the proximal end 14 to a taper point 20, and a distal shaft portion 22, which extends from the taper point 20 to the distal end 16. As shown in FIG. 1, the proximal shaft portion 18 has an outer diameter which is larger than that of the distal shaft portion 22.

Proximal end 14 is configured to be fitted with one or more connectors 24 for connecting catheter 10 to a urine collection system, or for receiving a syringe tip for irrigation of the urethra. Connector 24 may include a standard luer connector, a funnel connector or any combination thereof. In one embodiment, the distal shaft portion 22 of catheter body 10 includes a coude tip 26 having a length which extends from a bend 28 to the distal end 16 at an angle relative to the remaining distal portion 22. In one embodiment, coude tip 26 has a length of about 0.83 inches.

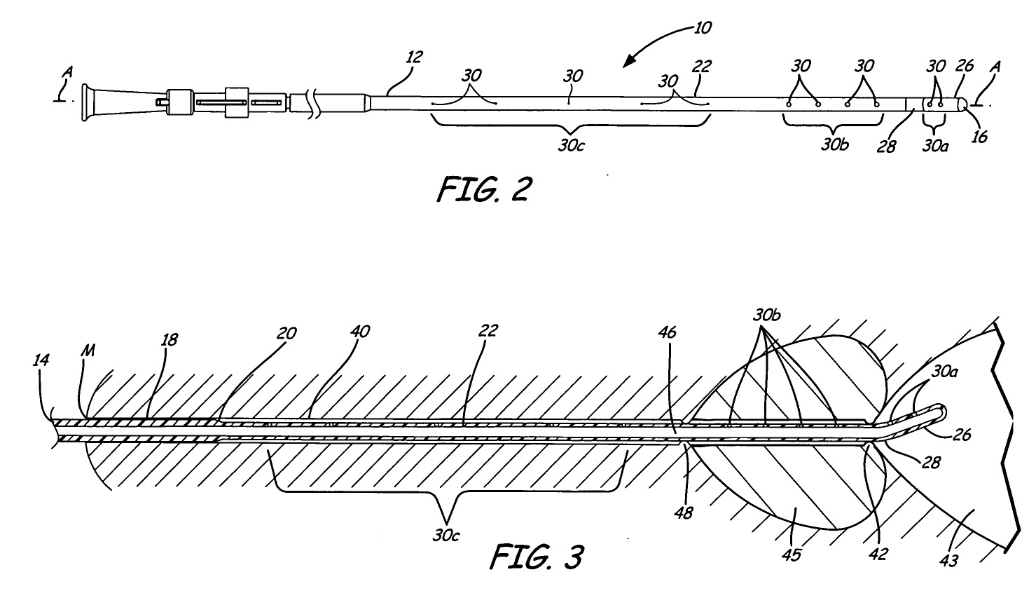

FIG. 2 is a top plan view of catheter 10 with the coude tip 26 elevated and oriented in a plane common with the distal shaft portion 22. As shown in FIGS. 1 and 2, distal shaft portion 22 is provided with a plurality of openings or holes 30 that communicate with an interior of shaft 12. In general, holes 30 are generally located along one side of distal shaft portion 22. In one embodiment, holes 30 are spaced longitudinally along one side of distal shaft portion 22 to be generally oriented in a common plane that bisects the longitudinal axis A of catheter body 12. A first set of spaced holes 30a are located in the coude tip 26 between bend 28 and distal end 16. A second set of spaced holes 30b are located proximal and generally adjacent to bend 28. A third set of spaced holes 30c are spaced proximally from the second set of holes 30b. In one embodiment, each of the first, second and third sets of holes 30a, 30b, 30c comprises at least two holes. In general, in one embodiment one or more of holes 30c have a diameter less than the diameter of holes 30b and 30c.

The following hole dimension and locations are representative of one embodiment of the invention. Each hole in the first and second set of holes 30a and 30b has a diameter of about 0.058 inches, and each hole in the third set of holes 30c have a diameter of about 0.021 inches. The first set of holes 30a comprises two holes that are spaced from distal end 16 by about 0.346 inches and 0.496 inches, respectively. The second set of holes 30b comprise four holes that are spaced apart from each other by about 0.40 inches, with the hole 30b closest to distal end 16 spaced from distal end 16 by about 1.170 inches to about 1.270 inches. The third set of holes 30c comprise five holes that are spaced apart from each other by about 1.0 inch, with the hole 30c closest to distal end 16 spaced from distal end 16 by about 3.320 inches to about 3.420 inches.

FIG. 3 is a longitudinal sectional view of one embodiment of catheter 10 positioned within a diagrammatic view of a male urethra 40 as viewed from the perspective of a patient in the supine position. As shown in FIG. 3, in one embodiment, catheter 10 is inserted in urethra 40 until the bend 28 of coude tip 26 extends distal of the internal sphincter 42 of the bladderneck and holes 30a are is positioned within bladder 44. Catheter 10 is oriented with holes 30a oriented generally upward as shown in FIG. 3, opposite the direction of gravity. The proximal shaft section portion 18 may incorporate orientation markings to ensure proper orientation of catheter 10 in the urethra. Holes 30a allow urine within bladder 43 to enter central lumen 46 of catheter 10 and flow to the proximal end 14.

As further shown in FIG. 3, holes 30b are located along the distal shaft portion 22 proximal of the bend 28 such that they are positioned adjacent to the prostate 45 between the external sphincter 48 and the internal sphincter 42. Holes 30c are located along the distal shaft portion 22 to be longitudinally spaced between taper point 20 of catheter body 12 and the external sphincter 48. In one embodiment, taper point 20 is located along catheter body 12 so as to be approximately 2.0 inches from the meatus tip M when coude tip 26 is positioned within bladder 43. Holes 30b and 30c are also oriented upward as shown in FIG. 3. Because of the orientation of holes 30a, 30b, 30c along a surface of shaft 12 in a common plane and the relatively low pressure of urine draining through lumen 46, urine flow is principally directed to proximal end 14 of catheter 10.

As shown in FIG. 3, the outer diameter and cross-sectional surface area of distal shaft portion 22 is selected to be less than that of the urethra 40, while the proximal shaft portion 18 is sized to substantially engage the entire circumference of the wall of urethra 40. In one embodiment, proximal shaft portion 18 has a diameter that is at least 1 French larger than that of distal shaft portion 22. By way of example, in one embodiment, the outer diameter of proximal shaft portion 18 is 18 French (i.e., 6.0 mm) and the outer diameter of distal shaft portion 22 is 14 French (i.e., 4.7 mm), and the inner diameter of central lumen 46 remains constant the full length of catheter 10. In one embodiment the inner diameter of central lumen 46 is about 0.125 inches.

As further shown in FIG. 3, once the bladder is drained, a fluid irrigant (e.g., an anesthetic) may be injected into catheter 10 at proximal end 14 via a connector 24 (not shown) to irrigate urethra 40. As the irrigant enters and flows through lumen 46, it reaches holes 30c first and begins to flow out of holes 30c. The remainder of the irrigant flows to holes 30b and begins to exit holes 30b. Because the outer surface of the proximal shaft portion 18 is in relatively close contact with to urethra 40, irrigant exiting holes 30c becomes trapped generally between taper point 20 and the external sphincter 48. Likewise, irrigant exiting holes 30b becomes trapped generally between external sphincter 48 and internal sphincter 42. In one embodiment, the diameter of holes 30c is selected such that irrigant flows through holes 30c at a slower rate than that of irrigant that flows through holes 30b thereby permitting a generally equal distribution of irrigant along urethra 40. In an alternative embodiment, the diameter of holes 30c can be substantially the same as the diameter of holes 30a and 30b. Additional irrigant can also be delivered to the bladder via holes 30a by continuing to inject irrigant into catheter 10 once urethra 40 becomes full. When the urethra or the urethra/bladder has been irrigated, catheter 10 is withdrawn and the treatment catheter may be inserted into the urethra for the urological procedure with minimal discomfort to the patient.

The present invention is a multi-functional, single lumen urological catheter that allows the bladder to be drained and the full length of the urethra to be irrigated with a single catheterization procedure. In one embodiment, the outer diameter of the distal shaft portion of the catheter, which extends a majority of the urethral length, has less surface area in contact with the urethral wall, while the larger outer diameter of the proximal shaft portion, which extends a relatively short distance within the urethra, provides an effective seal of the urethra. As a result, the catheter of the present invention is easily inserted the length of the urethra to the bladder with minimal discomfort to the patient.

A plurality of longitudinally spaced holes are formed in the catheter wall and are oriented on one side of the catheter in a plane that passes through the longitudinal axis of the catheter. The plurality of holes are positioned to be along a surface of the catheter opposite the direction of gravity when the catheter is inserted in a patient in the supine position. A first set of holes in the coude tip allows urine to drain from the bladder via a single central lumen of the catheter when the catheter is inserted in the patient. A second set of holes is located on the catheter proximal to the coude tip so as to be generally positioned between the internal and external sphincters when the coude tip is located within the bladder. A third set of holes is located on the catheter so as to be proximal to the external sphincter when the coude tip is positioned in the bladder. In one embodiment, the diameter of at least one of the holes in the third set of holes is selected to be smaller than the diameter of the holes in the first and second sets of holes. The holes of the second and third sets of holes allow an irrigant to be relatively evenly delivered and distributed to the full length of the urethra via the central lumen of the catheter. The size and spacing of the plurality of holes can be varied to adjust the flow and placement of the irrigant. The present invention thereby gently irrigates the entire urethra with relatively low irrigation pressure in comparison to irrigation techniques that attempt to irrigate the full length of the urethra by injecting irrigant at the meatus.

The proximal shaft portion provides a seal between the catheter and the urethra to minimize irrigant from leaking out of the urethra at the meatus as the urethra is being irrigated. Alternative configurations to the proximal shaft portion can be made to aid in creating a seal of the catheter relative to the urethra. By way of example, circumferential o-ring-style ribs or an inflatable balloon may be provided on the outer surface of the proximal shaft portion. In addition, an external clamp may be used. Although the present invention has been described with reference to exemplary embodiments, workers skilled in the art will recognize that changes may be made in form and detail without departing from the scope and spirit of the invention.

What is claimed is:

1. A urethral catheter comprising:
an elongate tubular shaft having an open proximal end, a sealed distal end, an outer surface and a single lumen extending between the proximal and distal ends, the tubular shaft having a plurality of openings communicating between the outer surface and the lumen, the plurality of openings being longitudinally spaced relative to one another and oriented on one side of the shaft in a common plane that bisects a longitudinal axis of the shaft, the plurality of openings comprising a first set of openings adjacent to the distal end of the shaft, a second set of openings spaced proximally of the first set of openings, and a third set of openings proximally spaced from the second set of openings, the third set of openings distally spaced from the proximal end of the shaft, wherein the plurality of openings comprise the only openings of the catheter.

2. The urethral catheter of claim 1 wherein at least one opening of the third set of openings has a diameter smaller than that of the openings of the first and second set of openings.

3. The urethral catheter of claim 1 wherein the tubular shaft further comprises a proximal shaft portion and a distal shaft portion, the distal shaft portion having an outer diameter less than an outer diameter of the proximal shaft portion, wherein the plurality of openings are located along the distal shaft portion of the catheter.

4. The urethral catheter of claim 3 wherein the distal shaft portion further comprises a tip portion adjacent to the distal end, the tip portion extending at an angle relative to the distal shaft portion, the first set of openings being located in the tip portion.

5. The urethral catheter of claim 1 wherein the first set of openings comprises first and second openings, the second set of openings comprises third and fourth openings, and the third set of openings comprises fifth and sixth openings, wherein at least one of the fifth and sixth openings has a smaller diameter than that of the third and fourth openings.

6. The urethral catheter of claim 5 wherein the third and fourth openings have a diameter of about 0.058 inches and the fifth and sixth openings have a diameter of about 0.021 inches.

7. The urethral catheter of claim 6 wherein the first and second openings are spaced relative to one another by about 0.15 inches, third and fourth openings are spaced relative to one another by about 0.40 inches, and the fifth and sixth openings are spaced relative to one another by about 1.0 inch.

8. The urethral catheter of claim 6 wherein the first opening is spaced relative to the distal end of the shaft by about 0.361 inches, the third opening is spaced relative to the distal end of the shaft by about 1.170 inches to about 1.270 inches, and the fifth opening is spaced from the distal end of the shaft by about 3.320 inches to about 3.420 inches.

9. A urethral catheter comprising:
an elongate tubular shaft having an open proximal end, a sealed distal end, an outer surface and a single lumen extending between the proximal and distal ends, the tubular shaft having a first shaft portion extending from the proximal end to a transition point, the first shaft portion having a first outer diameter, the catheter shaft further having a second shaft portion extending from the transition point to the distal end, the second shaft portion having a second outer diameter, the second shaft portion further having a tip portion adjacent to the distal end, the tip portion extending at an angle relative to the second shaft portion, the outer surface of the second shaft portion comprising a first plurality of holes in the tip portion, a second plurality of holes spaced from the first plurality of holes in a direction toward the proximal end, and a third plurality of holes spaced from the second plurality of holes in a direction toward the proximal end, the first, second and third plurality of holes communicating between the outer surface of the catheter shaft and the lumen and being located on one side of the catheter shaft and aligned in a common plane that bisects a longitudinal axis of the shaft, at least one of the holes of the third plurality of holes having a diameter less than that of each hole of the first and second plurality of holes.

10. A urethral catheter comprising an elongate tubular shaft having an open proximal end, a sealed distal end, an outer surface and a single lumen extending between the proximal and distal ends, the shaft having a proximal shaft portion, a distal shaft portion extending from the proximal shaft portion and a tip portion extending from the distal shaft portion and adjacent the distal end, the tip portion configured to be located in a bladder of a patient when the urethral catheter is inserted into a urethra of the patient, the tip portion comprising first and second openings spaced from the distal end, the first and second openings located along one side of the catheter and oriented in a plane that bisects a longitudinal axis of the catheter, the distal shaft portion having third, fourth, fifth and sixth openings located along the one side of the catheter and oriented in the plane that bisects the longitudinal axis of the catheter, the third and fourth openings being spaced longitudinally relative to one another so as to be located between an internal sphincter and an external sphincter of the patient when the tip portion is located in the bladder, the fifth and sixth openings longitudinally spaced relative to the fourth opening so as to be located between the external sphincter and a meatus of the patient when the tip portion is located in the bladder, at least one of the fifth and sixth openings having a diameter smaller than that of the third and fourth openings, wherein the first, second, third, fourth, fifth and sixth openings communicate with the single lumen of the shaft.

11. The urethral catheter of claim 10 wherein the proximal shaft portion has a first outer diameter and the distal shaft portion has a second outer diameter less than the first outer diameter.

12. The urethral catheter of claim 11 wherein the single lumen has an inner diameter, the inner diameter of the lumen in the proximal shaft portion being substantially the same as the inner diameter of the lumen in the distal shaft portion.

13. The urethral catheter of claim 10 wherein the first opening is spaced relative to the distal end of the shaft by about 0.361 inches, the third opening is spaced relative to the distal end of the shaft by about 1.170 inches to about 1.270 inches, and the fifth opening is spaced from the distal end of the shaft by about 3.320 inches to about 3.420 inches.

14. A method of preparing a urethra of a person for a transurethral urological procedure, the method comprising:
providing a urological catheter comprising elongate tubular shaft having an open proximal end, a sealed distal end, an outer surface and a single lumen extending between the proximal and distal ends, the shaft further having a plurality of longitudinally spaced openings communicating between the outer surface and the single lumen and oriented on one side of the shaft in a common plane that bisects a longitudinal axis of the shaft, the plurality of openings comprising the only openings of the catheter;
inserting the catheter into the urethra until a first opening of the plurality of openings is in communication with a bladder of the person, a second and third opening of the plurality of openings is positioned between an external sphincter and an internal sphincter of the person, and a fourth and fifth opening of the plurality of openings is positioned between the external sphincter and a meatus of the person, the proximal end extending external to the meatus;
draining urine from the bladder;
injecting a fluid into the proximal end of the shaft until the fluid exits the third, fourth, fifth and sixth openings, the fluid exiting the fifth and sixth openings at a first rate, the fluid exiting the third and fourth openings at a second rate greater than the first rate.

15. The method of claim 14 and further comprising:
sealing a proximal portion of the shaft relative to the urethra.

16. The method of claim 14 and further comprising:
injecting the fluid into the proximal end of the shaft until the fluid exits the first opening.
